(19) United States
(12) Patent Application Publication
(10) Pub. No.: US 2017/0182305 A1
(43) Pub. Date: Jun. 29, 2017

(54) CONNECTOR DISINFECTION SYSTEM

(71) Applicant: PURACATH MEDICAL, INC., San Francisco, CA (US)

(72) Inventors: James R. KERMODE, Los Altos, CA (US); Julia A. RASOOLY, San Francisco, CA (US)

(73) Assignee: PURACATH MEDICAL, INC., San Francisco, CA (US)

(21) Appl. No.: 15/301,905

(22) PCT Filed: Apr. 10, 2015
(86) PCT No.: PCT/US15/25352

§ 371 Date: Oct. 4, 2016

Related U.S. Application Data
(60) Provisional application No. 61/978,556, filed on Apr. 11, 2014.

(51) Int. Cl. A61M 39/16; A61M 39/18; A61M 1/14; A61L 2/10

(52) U.S. Cl. CPC A61M 39/16; A61L 2/10; A61M 39/18; A61M 1/14; A61L 2202/24; A61L 2202/14; A61M 2039/167; A61M 2205/8206; A61M 2205/502

(57) ABSTRACT

Devices, systems, and methods for disinfecting catheters used during in line catheter connections are provided. A disinfection device including one or more LED UV sources comprises a small volume kill zone. The disinfection device is configured to effectively disinfect fluid within the kill zone.

### INCORPORATION BY REFERENCE

All publications and patent applications mentioned in this specification are herein incorporated by reference to the same extent as if each individual publication or patent application was specifically and individually indicated to be incorporated by reference.

### FIELD

This application relates generally to sterilization of connectors, more particularly connectors such as central venous catheters (CVC) and peripheral internal central catheters (PICC) used in a medical application, for example during hemodialysis (HD), transmission of saline, dialysate, chemotherapy medications or other drugs.

### BACKGROUND

The current HD catheter connection process is a time-consuming and tedious process that depends upon strict nurse compliance for effectiveness. The process generally includes a thorough hand washing and gloves and facemasks for the nurse; a 2 minute alcohol scrub around the connection between the central venous catheter (CVC) and the tube that goes to the hemodialysis machine or other fluids; further use of disinfectants on the connectors; and a gentamycin and citrate flush in the line.

The above described procedure involves various materials and time, which cost hospitals and other care facilities a tremendous amount of financial resources. The procedure is also rigorous and requires various steps. Skipping or improper performance of just one of the steps can lead to catheter related blood stream infection (CRBSI). CRBSAI can result in increased complications for the patient and costly disruptions for the hospital or care facility.

Current products used in this area include needleless connectors such as the Tego® connector system. The Tego® connector uses a silicone seal to close the fluid path when the connector is not activated. The system is described in U.S. Pat. No. 7,497,849, filed May 2, 2006, the disclosure of which is hereby incorporated by reference.

UV disinfection systems are known in the art. U.S. Pat. Nos. 4,882,496; 7,834,328; 4,620,845; 6,461,568 and U.S. Publication Nos. 2005/0013729 and 2007/0274879, the disclosures of which are incorporated by reference herein in their entireties, describe such systems. A majority of such systems utilized Mercury Vapor lamps to provide the UV light. Mercury Vapor lights can require a relatively high operating voltage. UV LEDs can be used as an alternative to Mercury Vapor lamps. U.S. Pat. No. 8,469,545, filed May 10, 2012 and incorporated by reference herein, in its entirety, describes embodiments of UV LED sterilization systems. While UV disinfection systems utilizing LEDs have been disclosed in the prior art, such systems have not been described in commercially viable applications.

Through a great deal of research and visiting local dialysis clinics, the product presented here seeks to create a way to reduce costs and decrease the incidence of infections in catheter line connections (e.g., such as those used during hemodialysis or chemotherapy) patients using UV LEDs in a manner that is commercially viable.

### SUMMARY OF THE DISCLOSURE

In some embodiments, a disinfection device for use during catheter line connections is provided. The device comprises 2 LED UV sources configured to provide a kill zone comprising a volume less than about 1 cm³.

The catheter line connection can comprise a catheter used during dialysis or chemotherapy. The device can comprise a volume of less than about 41 cm³. The device can comprise a battery power source. The battery can comprise a coin cell battery. A lifespan of the device can be about 5 years. The device can comprise indicators showing disinfection status. The device exterior can comprise soft touch material. Each UV LED can comprise a UVC output of about 0.35-0.65 mW at about 30 mA. Each UV LED can comprise a spot diameter of about 0.5-0.8 cm.

In some embodiments, a disinfection device for use during catheter line connections is provided. The device comprises 2 LED UV sources configured to provide about 1.4-1.6 mW/sqcm.

The catheter line connection can comprise a catheter used during dialysis or chemotherapy. The device can comprise a volume of less than about 41 cm³. The device can comprise a battery power source. The battery can comprise a coin cell battery. A lifespan of the device can be about 5 years. The device can comprise indicators showing disinfection status. The device exterior can comprise soft touch material. Each UV LED can comprise a UVC output of about 0.35-0.65 mW at about 30 mA. Each UV LED can comprise a spot diameter of about 0.5-0.8 cm.

In some embodiments, a disinfection system for use during catheter line connections is provided. The system comprises a hub comprising a first opening, a second opening, and a third opening; the first opening comprising a first connector, the second opening comprising a second connector, and the third opening comprising a third connector, the hub comprising a valve proximate to the third opening, the valve configured to control flow between the third opening and first and second openings; a first tube comprising a first tube end, the first tube end comprising a first tube connector, and a flow controller; a second tube comprising a second tube end, the second tube end comprising a second tube connector, and a flow controller; and a disinfection device comprising at least one LED UV source and a kill zone comprising a small volume, wherein the first connector is configured to connect to the first tube connector and the second connector is configured to connect to the second tube connector, and wherein the hub, when connected to the first and second tubes, is configured to be positioned in the kill zone.

The hub can comprise features configured to mate with features of the disinfection device. The device can be configured to hold the hub. The device and the hub can provide a kill zone of less than about 1 cm³.

In some embodiments, a method for disinfecting catheters is provided. The method comprises closing a first flow controller positioned within a first tube; closing a second flow controller positioned within a second tube; connecting a first tube connector of the first tube to a first connector positioned at a first opening of a hub; connecting a second tube connector of the second tube to a second connector positioned at a second opening of the hub; connecting a syringe to a third connector at a third opening of the hub; opening a valve positioned in the hub near the third opening, the valve controlling flow between the third opening and the first and second openings; opening the first flow controller; drawing fluid into the syringe; closing the first flow controller; opening the second flow controller; drawing fluid into the syringe; closing the second flow controller; attaching a disinfection device at least partially around the hub, the first tube, and the second tube, the disinfection device comprising at least one LED UV source; and irradiating the hub with UV light using the LED UV source.

The disinfection device can be a handheld device. Drawing fluid into the syringe can minimize the area of potential contamination. Irradiating the hub with UV light can comprise activating the disinfection device for about 10-30 seconds. The first tube of second tube can be a central venous catheter (CVC). A patient with the implanted CVC can wear the hub connected to the CVC. The first or second tube can be a catheter. A patient can wear the hub attached to a distal end of the catheter. The patient can wear the disinfection device attached around the hub.

In some embodiments, a catheter set for use during hemodialysis is provided. The catheter set comprises a central venous catheter comprising a first UV-transmissive connector and a valve positioned less than 1 cm from the first UV-transmissive connector; and dialysis tubing comprising a second UV-transmissive connector configured to connect to the first UV-transmissive connector and a valve positioned less than 1 cm from the second UV-transmissive connector.

In some embodiments, a catheter set for use during chemotherapy is provided. The catheter set comprises a peripherally inserted central catheter comprising a first UV-transmissive connector and a valve positioned less than 1 cm from the first UV-transmissive connector; and tubing comprising a second UV-transmissive connector configured to connect to the first UV-transmissive connector and a valve positioned less than 1 cm from the second UV-transmissive connector.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 illustrates an embodiment of a hemodialysis catheter set up.

FIG. 2 illustrates an embodiment of a disinfection device.

FIG. 3 illustrates a perspective view of an embodiment of a disinfection device.

FIG. 4 illustrates an embodiment of the UV enclosure geometry for each LED.

FIG. 5A illustrates an embodiment of a hub.

FIGS. 5B-5C illustrate embodiments of tubes configured to connect to the hub.

FIGS. 6A-6D illustrate an embodiment of a method of disinfecting tubes in connection with performing peritoneal dialysis.

FIG. 7A illustrates an embodiment of a disinfection device.

FIG. 7B illustrates an embodiment of a hub.

FIG. 7C illustrates an embodiment of the hub of FIG. 7B positioned within the disinfection device of FIG. 7A.

FIG. 7D illustrates a user wearing the embodiment of FIG. 7C.

### DETAILED DESCRIPTION

Disclosed herein are devices, systems, and methods for disinfecting catheter line connections, for example catheters used during dialysis (e.g., hemodialysis) or chemotherapy. Examples of such catheters can include a central venous catheter (CVC) and a peripherally inserted central catheter (PICC). CVCs are generally placed into a large vein in the neck (internal jugular vein), chest (subclavian vein or axillary vein) or groin (femoral vein). It is used to administer medication or fluids, obtain blood tests (specifically the “central venous oxygen saturation”), and measure central venous pressure. PICCs are generally inserted in a peripheral vein in the arm, such as the cephalic vein, basilic vein or brachial vein, and then advanced proximally toward the heart through increasingly larger veins, until the tip rests in the distal superior vena cava or cavoatrial junction.

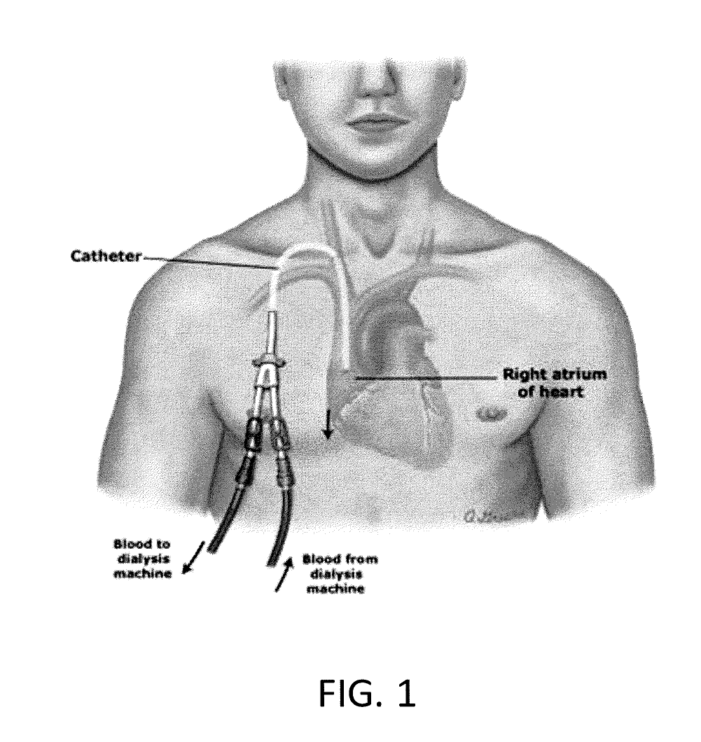

As described above, with respect to the example of hemodialysis in particular, hemodialysis can comprise connecting a CVC to dialysis machine. Hemodialysis is a renal replacement therapy generally conducted at a hospital or clinic. An intravenous catheter, arteriovenous fistula, or a synthetic graft can be used to gain access to the blood. As shown in FIG. 1, catheter access (e.g., using a CVC) generally includes a plastic catheter with two lumens which is inserted into a large vein (e.g., the vena cava through the internal jugular or femoral vein). Catheters (e.g., chronic dialysis catheters) can be used for hemodialysis if an AV fistula or graft is not tolerable for the patient. The catheter can be a permanent placement which is tunneled under the skin and inserted into the jugular or subclavian artery. Acute catheters can be used for temporary vascular access while the fistula or graft matures or the patient needs temporary dialysis immediately. Catheters used can be connected to and worn by the patient for up to 6 months. During dialysis, blood is withdrawn from one lumen, enters to dialysis circuit, and is returned via the other lumen. The connections between the catheter and the dialysis circuit must be clean in order to minimize the risk for infection. Instead of undergoing the burdensome sterile procedure described above involving various materials and requiring a significant amount of time, the connections can be made dirty and then cleaned through exposure to an amount of UV light sufficient to disinfect prior to beginning dialysis. This can provide a significant time advantage over existing systems (e.g., needlefree connections) that still require disinfection (e.g., using chemicals such as gentamycin flushes) prior to connecting.

The systems disclosed herein envision using a disinfection device comprising UV LEDs and configured to be positioned around catheter connections. The disinfection device can comprise a hand held device utilizing a low number (e.g., 2) of LEDs that are configured to provide a small volume kill zone, in which any bacteria present is exposed to sufficient UV energy to be killed. The area of the catheters/connectors to be disinfected can be configured to be disposed within the small kill zone of the disinfection device. The catheters used during HD can be configured for UV sterilization, comprising UV-transmissive materials. In other embodiments, the disinfection device can be used with one or more non-UV transmissive catheters, through the use of a UV transmissive hub, as described in more detail below. The UV transmissive hub can be configured to reduce the volume to be disinfected and concentrate the area of potential contamination into a small kill zone area.

The hub can be an adaptor configured to attach to a worn catheter (e.g., CVC catheter). The hub can advantageously be a universal adaptor configured to mate with any catheter design. Additionally, existing adaptors have a short lifespan. For example, the TEGO® system lasts for only 7 days. In contrast, the tee described herein can be connected to the catheter for up to 6 months, which should match the lifespan of the catheter.

The systems and methods described herein can significantly reduce the materials required for the hemodialysis procedure. For example, current disinfection protocol requires the use of gloves, gauze pads, face mask, saline syringe, saline flush, gentamycin/sodium citrate flush, and alcohol swabs. The current system uses a reusable hub and disinfection device, which can greatly reduce hospital or clinic costs. The cost savings can be about $488/yr/patient, assuming an HD patient receiving 3 treatments a week. For 500,000 patients for instance, that accounts for about $244M in savings. Additionally, the time saved by not having to perform the rigorous disinfection protocol significantly reduces hospital or clinic costs. Finally, prevention of additional disinfection also significantly reduces hospital or clinic costs.

U.S. Pat. No. 8,469,545, filed May 10, 2012 and incorporated by reference herein, in its entirety, describes embodiments of UV LED sterilization systems. While UV disinfection systems utilizing LEDs have been disclosed in the prior art, such systems have not been described in commercially viable applications. By utilizing a disinfection device comprising a small volume kill zone, along with catheters and connectors configured to comprise a small area to be disinfected, the systems disclosed herein provide commercially viable solutions for disinfection of connectors between conduits such as catheters. Previously described systems included large and/or a high number of LEDs. Such features can be prohibitively expensive to manufacture and cumbersome to use. In contrast, the system disclosed herein utilizes a small number of LEDs (e.g., 2) within a device comprising a small volume. Existing systems can also require a long exposure time for disinfection. The systems described herein can accomplish disinfection in less than a minute.

Relatedly, previously described systems involved large power requirements. In contrast, the disinfection device described above can require less power than traditional systems, allowing for simpler and smaller power supplies. For example, the system can utilize coin cell type batteries as the power source. The ability of the disinfection device to use fewer/smaller LEDs and run off less power than previously described systems allows for a disinfection device with a small form factor. As described in more detail below, the disinfection device can be golf-ball sized, which would enable it to be carried in a user's pocket, in some embodiments.

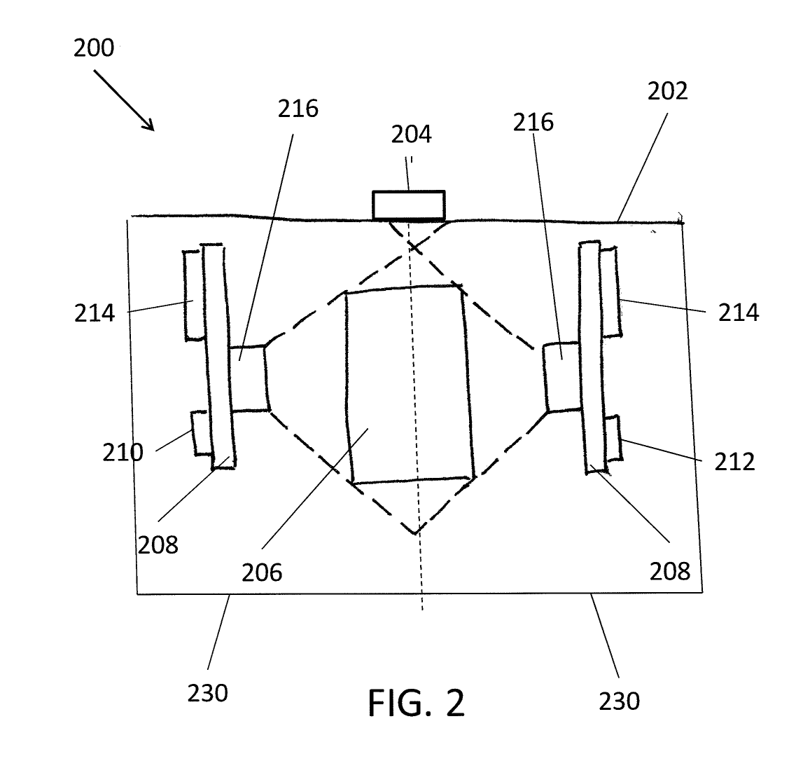

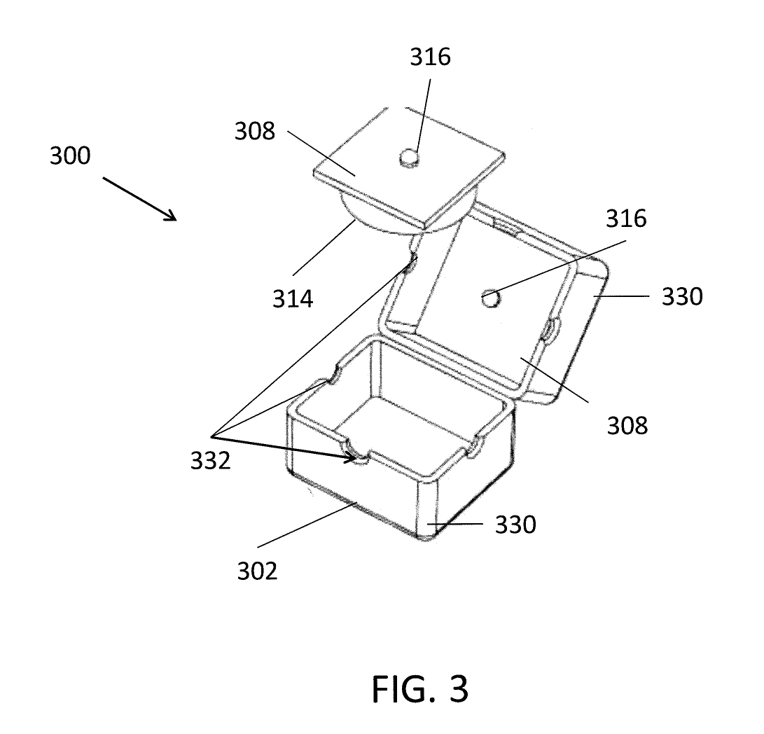

FIG. 2 illustrates a side cross-sectional view of an embodiment of a disinfection device 200. The disinfection device comprises a housing 202, partially shown in FIG. 2. The housing 202 can comprise a clamshell form, comprising two halves 230 configured to fold together and connected by a hinge 204. The hinge 204 can be, for example, a spring hinge. Other designs are also possible. For example, the two halves may not be hinged. The device is shown as rectangular in shape; however, other configurations are also possible. For example, the device can be disc shaped, kidney shaped, square shaped, or spherical. The device 200 can have sidewalls, as shown in FIG. 3. In some embodiments, the device 200 simply has a top and bottom portion configured to surround the portion of connectors to be disinfected.

The device 200 comprises a UVC LED 216 positioned at two sides (e.g., opposing sides, top and bottom, etc.) of the device 200. While the device 200 shows 2 UVC LEDs, it will be appreciated that more than 2 UVC LEDs can be used. For example, 3, 4, 5, 6, 7, 8, or more UVC LEDs can be used. In some embodiments, LED Model No. UVTOP-255TO39FW from Sensor Electronic Technology, Inc. can be used. The LED wavelength can be about 230-280 nm (e.g., about 235-275; about 245-270; about 255-260; or about 255 nm). The LED can comprise a spot diameter of about 0.5-0.8 cm (e.g., about 0.55-0.75; about 0.6-0.7; or about 0.65 cm) in diameter. The power consumption can be about 125-175 mW (e.g., about 130-170; about 140-160; about 145-166; or about 150 mW). The UVC output can be about 0.35-0.65 mW (e.g., about 0.4-0.6, about 0.45-0.55, about 0.50 mW) at about 30 mA (7.0V). The UVC power can be about 1.3-1.7 mW (e.g., about 1.4-1.6, about 1.45-1.55, or about 1.5 mW/cm²). The fluence of the LED can be >about 98% of the maximum output throughout a cone within 20° of the central axis of illumination. The dotted lines extending from the LEDs show the region in front of the LEDs illuminated by the light coming from the LEDs. Within those regions is kill zone 206. Within this zone 206, fluence can be sufficient to kill bio-contaminants present.

In some embodiments, kill zone 206 is about one cubic centimeter (about 1 cm×about 1 cm×about 1 cm). The width of the kill zone 206 can be about 0.5-1.5 cm. The length of the kill zone can be about 0.5-1.5 cm. The width of the kill zone 206 can be about 0.5-1.5 cm. Other sizes are also possible. For example, the kill zone 206 can have a volume of about 2 cm³, about 3 cm³, about 4 cm³, about 5 cm³, about 6 cm³, greater than 6 cm³. The kill zone 206 can be in the shape of a sphere. The device 200 can comprise an indicator to show that the area of the catheters/connectors to be disinfected is properly placed within the kill zone 206. For example, markings or coloring may distinguish the kill zone from other areas of the device. For another example, the kill zone can comprise features shaped to receive the catheters/connectors.

The device 200 comprises PCBs 208 positioned at both sides of the device 200. In some embodiments, the device 200 comprises 1 PCB. In some embodiments, the device 200 comprises more than two PCBs. The device 200 comprises a user control 210 (e.g., switch, push-button, etc.) connected to a PCB 208. In some embodiments, the device 200 comprises a user control for each PCB. The device 200 can comprise a timer 212. The timer 212 can be mounted to one of the PCBs 208. The timer 212 can be used for controlling power to the LEDs 216. The device comprises 3.6 volt, rechargeable coin cell batteries, such as Lir 2450 available from Powerstream, Orem, Utah 214. Other sources of power are also possible (e.g., other voltage coin cell batteries, AA batteries, AAA batteries, plug in power). Care facilities such as hospitals and clinics can have charging stations (e.g., similar to a home phone charging station), at which the disinfection units can charge. For example, one charging station can be provided for about 3-5 patient beds in an ICU unit or other units.

FIG. 3 illustrate an exploded, perspective view of an embodiment of a disinfection device 300. The device comprises a housing 302 with a clamshell form, comprising two halves 330 configured to fold together and connected by a hinge (not shown). FIG. 3 shows an LED 316 mounted above a PCB 308 which is positioned above a battery 314, all of which are configured to be positioned within the bottom half 330 of the device. An LED 316 and PCB 308 are also visible in the top half of the device 300 shown in FIG. 3. A second battery may be positioned above the top PCB 308.

Each half 330 comprises recesses 332 which form openings in the housing 302 when the device 300 is closed. The openings are configured to permit a portion of a connection between catheters (e.g., two catheters connected directly, two catheters connected through a hub) to be positioned within the device 300 and extend from the device 300. The device 330 is shown with three openings; however, more openings or fewer openings are also possible (e.g., 2, 4, 5, 6, etc.). It will be appreciated that the device 300 can comprise other features described with respect to FIG. 2, which are not shown in FIG. 3. (e.g., user control, timer, etc.).

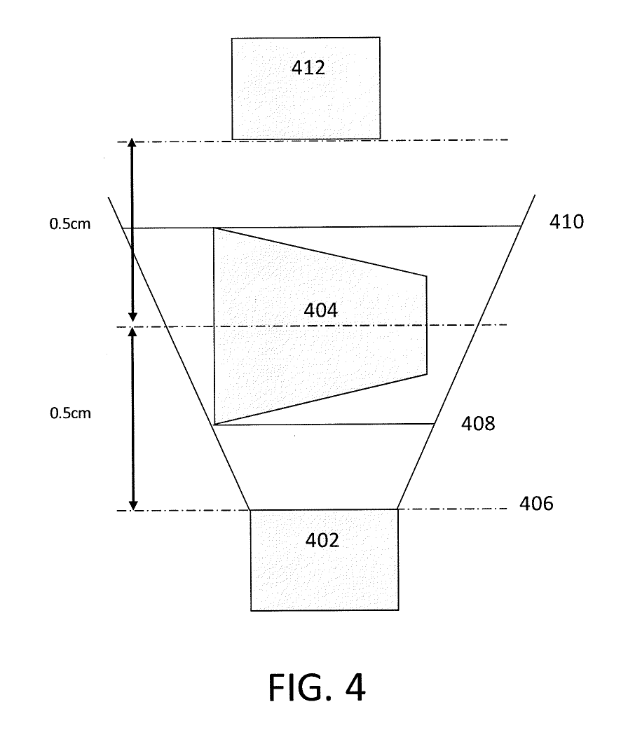

FIG. 4 illustrates the UV enclosure geometry for each LED, in some embodiments. The LED (e.g., single UVC LED, 216, as described with respect to FIG. 2) 402 is positioned about 0.5 cm from the central axis of the Luer connection 404. FIG. 4 illustrates the spot diameter of the light emission as it travels from the LED 402, within the high fluence cone of 20 degrees about the central axis of emission, towards and past the Luer connection, 404. FIG. 4 illustrates the spot diameter, 406, at the surface of LED 402 (for example 0.65 cm), the spot diameter, 408 (for example 0.85 cm), at the nearside of the Luer connection 404, and the spot diameter 410 (for example 1.20 cm) at the far side of the Luer Connection 404. FIG. 2 describes the light coming from a single LED, 402. However, there is a second LED, 412, 1.0 cm away from LED 402 (0.5 cm from the Luer connection axis). Table 1, below, provides the spot diameter and fluence at those various spots for each LED 402, and 412, and the combined LEDs 402 and 412. The fluence refers to the rate of energy intersecting a unit area. Table 1 also shows the unattenuated fluence (1.33 mW/sqcm). However, the light coming from LEDs 402 and 412 will be attenuated as it passes through the material comprising the Luer connection 404. If, for example, the Luer connection 404 is made from Mitsui Chemicals DX820, the attenuation will be about 45% per 0.2 cm of material. Taking this into account, as shown below, the lowest, attenuated fluence is 0. 73 mW/sqcm. The spot diameter and fluence at various distances from the LED(s) can be used to design the disinfection device 200 to help ensure sufficient fluence and exposure time and therefore sufficient disinfection of the conduits and connectors to be disinfected.

TABLE 1

| | |
|---|---|
| Spot Diameter (area) at UV | 0.65 cm (0.33 sqcm) |
| LED 402 (Table 1): | |
| Unattenuated Fluence at UV | 1.5 mW/sqcm |
| LED 402 (Table 1): | |
| Spot Diameter (area) at | 0.84 (0.56 sqcm) |
| Nearside Luer 408: | |
| Unattennuated Fluence at | 0.88 mW.sqcm (= 0.33 sqcm/ |
| Nearside Luer 408: | 0.56 sqcm*1.5 mW) |
| Spot Diameter (area) at | 1.2 cm (1.1 sqcm) |
| Farside Luer 410: | |
| Unattenuated Fluence at | 0.45 mW/sqcm (= 0.33 sqcm/ |
| Farside Luer 410: | 1.1 sqcm*1.5 mW) |
| Unattenuated Fluence (both LEDs | 1.33 mW/sqcm (= 0.88 mW/sqcm + |
| 402, 412) at Farside Luer 410: | 0.45 mW/sqcm) |
| Lowest, Attenuated Fluence | 0.73 mW/sqcm |
| (both LEDs 402, 412) | (= 0.55*1.33 mW/sqcm) |

The disinfection device can be powered by external or plug-in power. In some embodiments, the disinfection device can be powered by one or more batteries (e.g., 2, 3, 4, 5, etc.). In some embodiments, rechargeable coin cell batteries (e.g., Lir 2450 manufactured by Powerstream Technology) are used. In embodiments utilizing two such batteries, the batteries can have a nominal capacity of about 240 mAh (120 mAh for 2 batteries). The batteries can have a nominal voltage of about 7.2 V (3.6V per battery). The capacity for ≧7V at 60 mA draw can be about 108 mAh. The total usable energy per charge can be about 216 mAh. Other batteries are also possible. For example, in some embodiments, a Lir 2477, manufactured by Powerstream Technology can be used.

Table 2, below, shows the UVC exposure required for disinfection of various species of bacteria and viruses.

TABLE 2
Disinfection (3 Log reduction)

| Species | UVC Exposure (est.) |
|---|---|
| Candida albicans | 8 mJ/sqcm |
| Escherichia coli | 5 mJ/sqcm |
| Pseudomonas aeruginosa | 7 mJ/sqcm |
| Staphylococcus aureus | 10 mJ/sqcm |
| Staphylococcus epidermidis | 4 mJ/sqcm |

As shown in Table 2 above, the UVC exposure required for Staphylococcus aureus is 10 mJ/sqcm. Based on the fluence calculations above in Table 1, the time required for killing Staphylococcus aureus would be about 14 sec (calculation: 10 mJ/sqcm/0.73 mW/sqcm).

Table 3, below shows the lifespan for two LEDs (e.g., the LEDs described with respect to FIG. 2) using 2 coin cell batteries (e.g., batteries described with respect to FIG. 2) is 0.72 hours or 2592 seconds. Based on the lifespan and a 15 sec cycle time, the disinfection device can perform about 173 cycles before needing to be recharged.

TABLE 3

| | |
|---|---|
| LED Power Consumption n(2 LEDs): | 300 mW (150 mW × 2 LEDs) |
| Time to Recharge: | 0.72 hours (216 mAh/300 mW) |
| | 2592 sec (0.72 Hours × 3600 |
| | seconds) |
| Cycles to Recharge (15 sec/cycle): | 173cycles (2592 sec/15 sec/cycle) |

The disinfection device 300 can be about 3 cm×about 3 cm×about 4 cm. In some embodiments, a width of the device is about 1-5 cm or about 2-4 cm. In some embodiments, a length of the device is about 1-5 cm or about 2-4 cm. In some embodiments, a depth of the device is about 1-6 cm or about 3-5 cm. In some embodiments, the dimensions of the device are selected based on the size of the circuitry and batteries disposed therein. In some embodiments, the disinfection device comprises about the same volume as a golf ball (about 41 cm³).

As described above, the disinfection device comprises a small volume kill zone. The catheters and connectors used with the disinfection device can be specially configured to comprise an area to be disinfected sized to fit within this small volume kill zone. Connectors compliant to ISO 594 (Luer connectors) are also appropriately sized to fit within this kill zone. In some embodiments, UVC-transmissive catheters are used as the CVC or PICC catheter. Such catheters can comprise UV-transmissive connectors and valves/clamps spaced closely enough (e.g. valves spaced less than about 2 cm from one another) to the connectors to allow the disinfection device to fit within the kill zone.

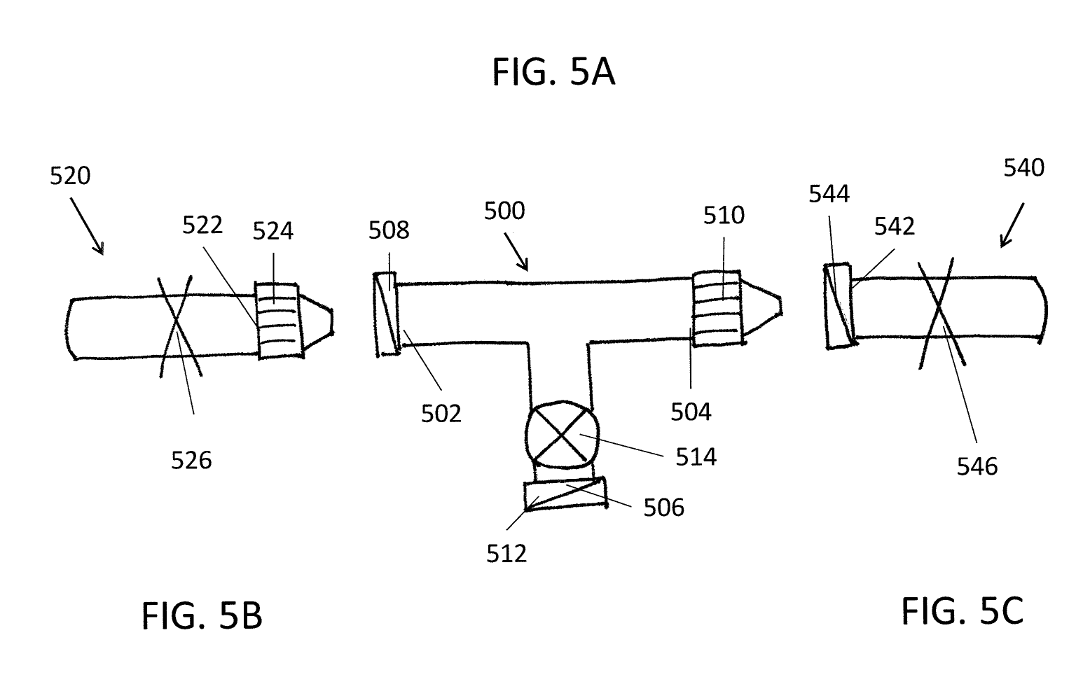

In other embodiments, the disinfection device can be used with one or more non-UV transmissive catheters. FIG. 5A illustrates an embodiment of a three-way hub 500 that can be used in such embodiments. The hub 500 can be configured to provide an area to be disinfected sized to fit within the kill zone 206 of the disinfection device. The hub comprises a body comprising three openings 502, 504, 506. The hub body can be, at least partially, UV transmissive. In some embodiments, the hub body is completely UV transmissive. The opening 502 comprises a connector 508. The connector can comprise a female Luer connector. The opening 504 comprises a connector 510, which can be a male Luer connector. The male Luer collar can comprise a UV-transmissive material. The opening 506 comprises a connector 512, which can be a female Luer connector. The hub serves as a sort of universal adaptor to a number of different catheter types (e.g., CVC, PICC) as the connectors (e.g. Luer) allow the hub to be attached to these different catheter types.

The hub 500 comprises a valve 514 positioned between opening 506 and openings 502, 504. The valve 514 can be Luer activated. In some embodiments, the valve 514 is integrated into the hub 500. In some embodiments, the valve comprises a flow control means configured to prevent flow from opening 506. The valve can comprise a UV-transmissive material. For example, in some embodiments, the valve comprises silicone.

The hub can be about 2 cm wide (across the top of the Tee), about 3 cm tall and about 1 cm deep. In some embodiments, the hub is about 1-3 cm wide. In some embodiments, the hub is about 2-4 cm tall. In some embodiments, the hub is about 0.5-1.5 cm deep.

FIG. 5B illustrates an embodiment of an end of tubing 520 (e.g., a conduit, a catheter) comprising an opening 522. The opening comprises a connector 524, which can be a male Luer lock connector. The tubing 520 includes a flow controller 526 for closing the tubing 520 leading to or feeding opening 522. The flow controller can comprise Luer style stopcocks, pinch clamps, roller clamps or tubing clamps. The tubing 540 can be part of an in line catheter circuit (e.g. a dialysis circuit, leading to a fluid bag, such as, dialysate bag, saline solution, chemotherapy or other drug).

FIG. 5C illustrates an embodiment of an end of tubing 540 (e.g., a conduit, a catheter) comprising an opening 542. The opening comprises a connector 544, which can be a female Luer lock fitting. The tubing 540 comprises a flow controller 546 for closing the tubing 544 leading to or feeding opening 542. The flow controller 546 can comprise Luer style stopcocks, pinch clamps, roller clamps or tubing clamps. The tubing 540 can be part of an in line catheter circuit (e.g. a dialysis circuit, leading to a fluid bag, such as, dialysate bag, saline solution, chemotherapy or other drug).

It will be appreciated that the connectors described herein as male or female can be the opposite, in some embodiments. For example, connectors described as male can be female. Connectors described as female can be male. The connectors can comprise ISO 594, Conical Luer Fittings. In some embodiments, the connectors comprise a 4-methylpentene-1 based polyolefin (e.g., DX820 available from Mitsui Chemicals America, Inc.). Other materials are also possible. For example, in some embodiments, the connectors comprise a fluoropolymer (e.g., EFEP TP-4020 or RP4040 available from Daikin Industries, Ltd.).

Additionally, in some embodiments, the connectors can be connector types other than Luer connectors. For example, barbed or slip fit connectors can be used.

Female Luer connectors can be formed integral to the tubing or component they are part of. For example, connector 508 and/or connector 512 can be molded integral to the hub 500.

Method of Using the Disinfection System with the Hub

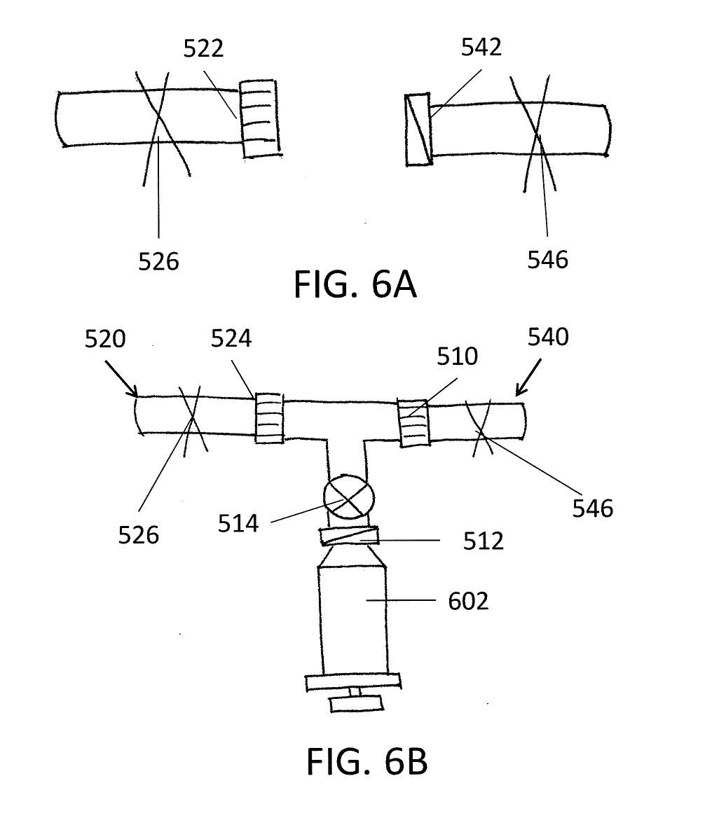

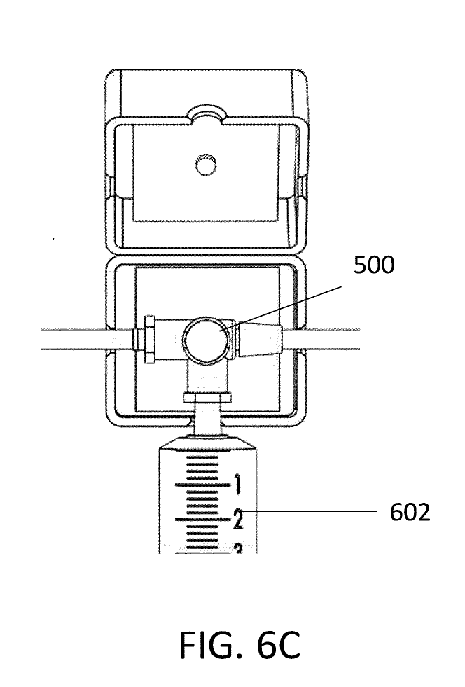

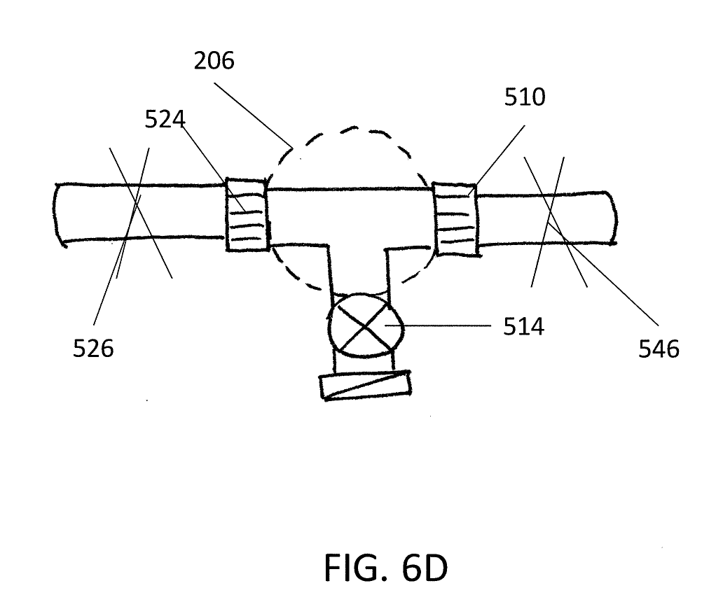

FIGS. 6A-6C illustrate an embodiment of a method for disinfecting the connection between two conduits. When using the system disclosed herein to disinfect tubing and connectors (e.g., the tubing and connectors described with respect to FIGS. 5A-5C), the flow controllers 526, 546 leading to openings 522, 542 are closed, as shown in FIG. 6A. Closing these tubes can limit how many and how far contaminants may travel into the tubes. The male connector 524 of tubing 520 can then be connected to female connector 508 (not shown) on the hub 500. The female connector 544 (not shown) of tubing 540 can be connected to male connector 510 on the hub 500. A syringe 602 is connected to the female connector 512 of the hub 500, as shown in FIG. 6B. The syringe 602 can be a 10 cc syringe, in some embodiments. Other syringe sizes are also possible. Connecting the syringe 602 to the connector 512 can automatically open the valve 514. In other embodiments, the valve is opened after connection of the syringe. The flow controller 526 is then opened. A small volume (e.g., about 2 cc, about 4 cc, about 6 cc) is drawn into the syringe 602. Flow controller 526 is closed. Flow controller 546 is then opened. A small volume (e.g., about 2 cc, about 4 cc, about 6 cc) is drawn into the syringe 602. Flow controller 546 is closed. It will be appreciated that while the description specifies that fluid is first drawn from conduit 520, in some embodiments, the fluid can first be drawn from conduit 540, and then drawn from conduit 520. The syringe 602 can then be removed and discarded. The valve 514 can close automatically upon removal of the syringe, in some embodiments. In other embodiments, the valve is then closed. In some embodiments, the syringe 602 is connected to the hub 610 while the disinfection device is already positioned within the hub 610, as shown in FIG. 6C. In such embodiments, the valve 514 is closed prior to irradiation. Drawing fluid down through the valve in this manner can help remove potential contamination from the in line catheter circuit and minimize the volume to be disinfected to ensure thorough disinfection.

A disinfection device (e.g., disinfection device 200) is clamped over the hub 500. The device can be clamped on before, during, or after the draw down. In some embodiments, the device is worn on the patient. The disinfection device 200 and the hub 500 are configured such that the kill zone 206 spans the area between connector 524, connector 510, and valve 514, as shown in FIG. 6D. The disinfection device can then be activated, for example, via user control (e.g., user control 210). The device can be activated such that it, at least partially, disinfects the hub 500 in zone 206. For example, the device can be activated for about 10-30 seconds (e.g., 15 seconds). In some embodiments, the device can be activated for a greater amount of time (e.g., about 10-180 seconds; about 60 seconds; about 120 seconds; or about 180 seconds).

In some embodiments, at least about 50% of the contaminants are killed. In some embodiments, at least about 60% of the contaminants are killed. In some embodiments, at least about 70% of the contaminants are killed. In some embodiments, at least about 80% of the contaminants are killed. In some embodiments, at least about 90% of the contaminants are killed. In some embodiments, at least about 99% of the contaminants are killed. In some embodiments, at least 99.99% of the contaminants are killed. In some embodiments, at least 99.99999% of the contaminants are killed.

The device 200 can then be unclipped (removed from hub 500) and stored and or recharged. The flow controllers 526, 546 can be opened. Flow can now be allowed through the clean connection. The device 200 and hub 500 can be re-used as desired to disinfect the connection between conduits 520, 540.

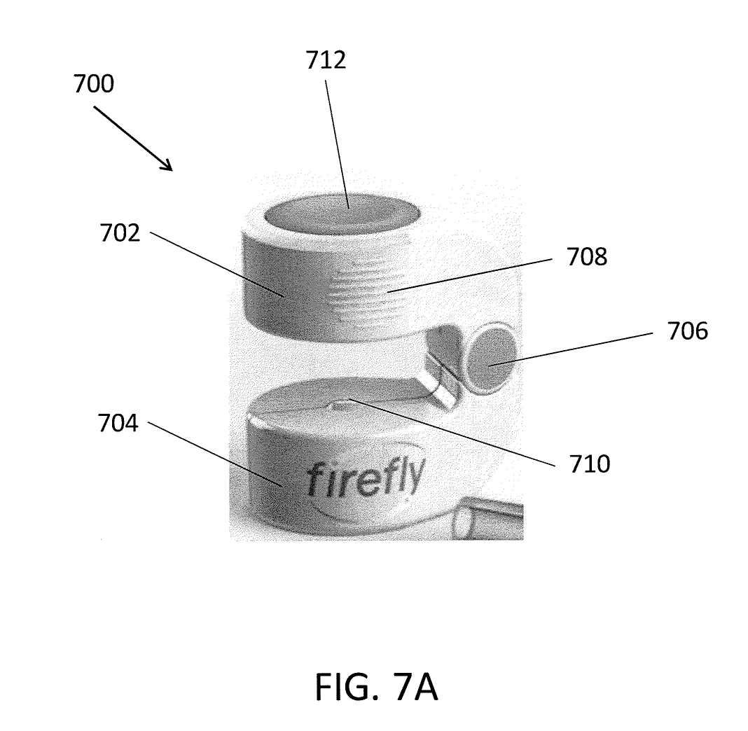

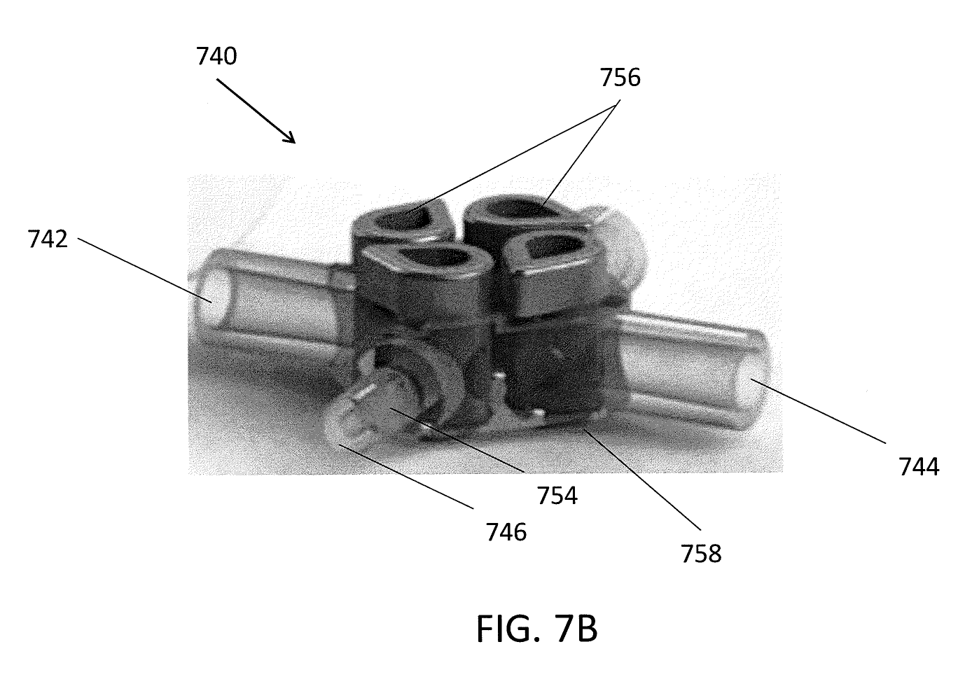

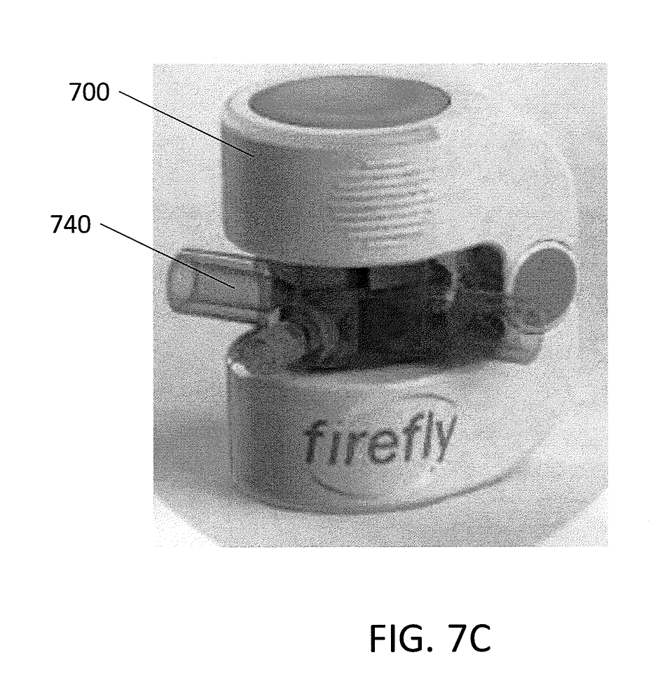

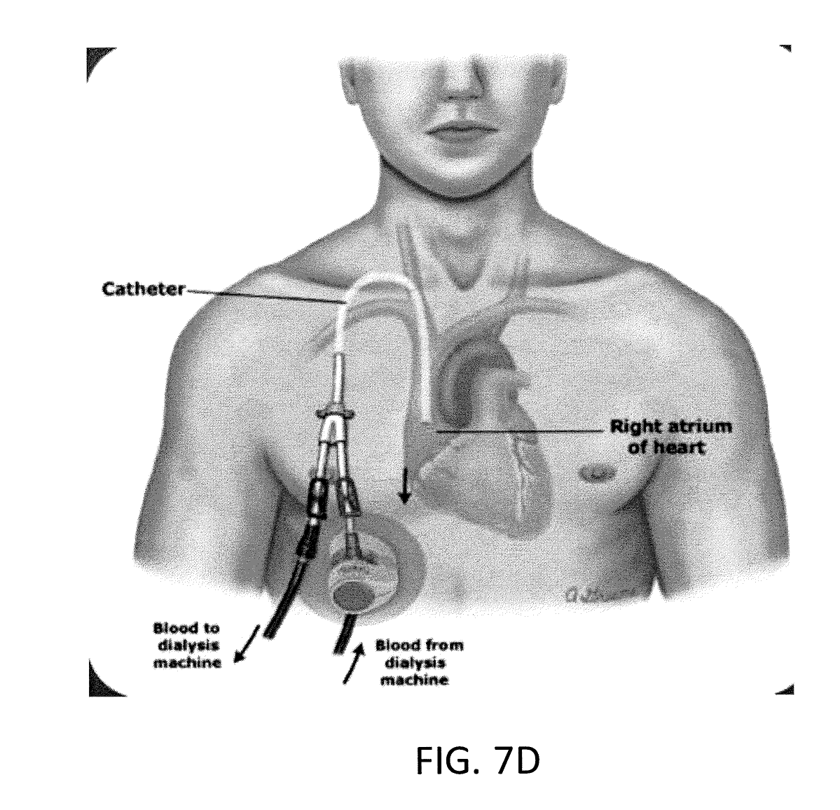

FIG. 7A illustrates another embodiment of a disinfection unit 700. Similar to the disinfection units 200, 300, the disinfection unit 700 comprises a top portion 702 and a bottom portion 704 connected by a hinge 706. The top or bottom portion can comprise a textured grip 708 to aid in grasping a portion of the unit to open or close the unit. Each of the top and bottom portion comprises components similar to those described above with respect to units 200, 300. Each portion includes at least one UVC LED 710, such as those described above. The unit 700 can comprise an area (e.g., logo area) backlit by the light source that can be used to indicate disinfection status (e.g., disinfected, error, etc.) of the unit 700. The unit 700 can comprise a power or activation button 712. The bottom of the unit 700 (not shown) can comprise a soft touch material configured to provide comfort to a user as the unit 700 can be worn by the user (FIG. 7C).

FIG. 7B illustrates another embodiment of a three-way hub 740. Unless otherwise specified, the hub 740 is similar to hub 500. The hub 740 includes three openings 742, 744, 746. The hub body can be, at least partially, UV transmissive. In some embodiments, the hub body is completely UV transmissive. The openings 742, 746 can comprise connectors. A valve 754 is positioned between opening 742 and openings 742, 744. The valve 754 can be Luer activated. In some embodiments, the valve is integrated into the hub. The valve 754 can comprise a flow control means configured to prevent flow from openings 746. The valve 754 can be configured to allow passage of a syringe, but to re-seal upon removal of the syringe. The hub comprises features 756 which can interact with features on the disinfection unit (not shown) to help hold the hub within the disinfection unit 700. The features 756 can also be used to provide a soft surface to the hub 700, making it more comfortable for a user to wear the hub. Inner surface of the features can comprise reflectors configured to concentrate UV light on the area to be disinfected. The bottom surface 758 of the hub can also provide a soft surface for user comfort. The features 756 can also be used to focus the UV light from the disinfection unit 700 onto a small kill zone area positioned within the features 756. The kill zone can extend beyond the features 756. As the disinfection unit 700 does not have sidewalls, the features, 756 can be used to concentrate UV light on the area to be disinfected.

FIG. 7C illustrates an embodiment of the disinfection unit 700 clamped over the hub 740. The UV lights 710 are centered above and below the kill zone area. The hub 740 and disinfection unit 700 can be used as described in the method above. FIG. 7D illustrates an embodiment of a patient wearing the unit 700 and hub 740. The unit 700 and hub 740 can be worn as shown in FIG. 7D during dialysis or between dialysis sessions (or similar transmission of other fluids in and out of the patient from an external source or bag). The open sides of the unit 700 allow access to the openings 702, 704, 706.

The disinfection unit can comprise a closed sensor. The sensor can provide feedback indicating a closed position to an internal monitoring system. The unit can be configured to be disabled and not provide UV disinfection energy until the unit is closed (clamped down) and in the right position with all other ports in the correct configuration. Other sensors are also possible. For example, hall effect, optical or a microswitch can be used.

The disinfection unit can comprise a control panel. In some embodiments, the control panel includes an activation button and a power button. The control panel can comprise a flex panel overlay which can provide protection from water ingress and provide UV blocking functionality. The control panel can provide feedback regarding the device state. For example, a green light and/or a beep can indicate that disinfection is complete. A red light can indicate error and prompt a user to redo the disinfection. The control panel can be backlit by the UV light source of the device. This UV backlighting can allow a user to verify UV output and that the disinfection process has completed or is happening. If the user takes off the firefly prematurely, there should be an alarm of some sort with different number of beeps or sound to notify the patient and care provider that the disinfection cycle is not complete and that they need to re-do the process.

The body of the disinfection unit can comprise a UV opaque material such as polycarbonate, ABS or polyethylene. The body can act as a light block to prevent UV light from travelling outside the unit and for protection against the patient.

Although the device has generally been described with respect to hemodialysis, it will be appreciated that it can also be used in other applications (e.g., peritoneal dialysis, other catheter applications including PICC lines, AV fistulas, portacath or chemotherapy applications, urinary catheters, etc). The hub described herein can be used with other disinfection units, such as that described in U.S. Provisional Application No. 62/052,164, filed Sep. 18, 2014, the disclosure of which is hereby incorporated by reference in its entirety. Additionally, the disinfection unit described herein can be used with other connector systems such as that described in U.S. Provisional Application No. 62/008,433, filed Jun. 5, 2014, the disclosure of which is hereby incorporated by reference in its entirety.

Variations and modifications of the devices and methods disclosed herein will be readily apparent to persons skilled in the art. As such, it should be understood that the foregoing detailed description and the accompanying illustrations, are made for purposes of clarity and understanding, and are not intended to limit the scope of the invention, which is defined by the claims appended hereto. Any feature described in any one embodiment described herein can be combined with any other feature of any of the other embodiment whether preferred or not.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1-35. (canceled)

36. A disinfection device for use during catheter line connections, the device comprising
6 LED UV sources configured to provide a kill zone comprising a volume less than about 3 cm³, the kill zone comprising features configured to receive catheter line connectors.

37. The disinfection device of claim 36, wherein the catheter line connection comprises a catheter used during dialysis or chemotherapy.

38. The disinfection device of claim 36, comprising a battery power source.

39. The disinfection device of claim 36, wherein a lifespan of the device is about 5 years.

40. The disinfection device of claim 36, comprising indicators showing disinfection status.

41. The disinfection device of claim 36, comprising indicators showing errors.

42. The disinfection device of claim 36, comprising indicators showing low battery.

43. The disinfection device of claim 36, wherein an exterior of the device comprises soft touch material.

44. The disinfection device of claim 36, wherein the catheter line connection comprises a central venous catheter (CVC).

45. A method for disinfecting catheter line connections, the method comprising
placing a catheter line connection into a disinfection device comprising 6 LED UV sources configured to provide a kill zone comprising a volume less than about 3 cm3, the kill zone comprising features configured to receive catheter line connectors; and
activating the disinfection device to disinfect the volume of the catheter line connection contained within the kill zone.

46. The method of claim 45, wherein the disinfection device is a handheld device.

47. The method of claim 45, wherein activating the disinfection device comprises activating the disinfection device for about 10-30 seconds.

48. The method of claim 45 wherein the catheter line connection comprises a central venous catheter (CVC).

49. The method of claim 45, wherein the patient wears the disinfection device.

50. A catheter set for use during hemodialysis or chemotherapy, comprising
a catheter central venous catheter comprising a first UV-transmissive connector and a valve positioned less than 1 cm from the first UV-transmissive connector and connected to the first UV-transmissive connector; and
infusion tubing comprising a second UV-transmissive connector configured to connect to the valve.
